(19) United States
(12) Patent Application Publication
(10) Pub. No.: US 2007/0225644 A1
(43) Pub. Date: Sep. 27, 2007

(54) Embolic removal for orthopedic procedures

(76) Inventor: Peter Berger, Danville, PA (US)

Correspondence Address: DARDI & ASSOCIATES, PLLC, 220 S. 6TH ST., SUITE 2000, U.S. BANK PLAZA, MINNEAPOLIS, MN, 55402, US

(21) Appl. No.: 11/726,206

(22) Filed: Mar. 8, 2007

Related U.S. Application Data
(60) Provisional application No. 60/780,227, filed on Mar. 8, 2006.

(51) Int. Cl. A61M 29/00

(52) U.S. Cl. 604/102.01

(57) ABSTRACT

Certain embodiments are a method of treating a patient comprising deploying an embolic trap to remove emboli from a vein of the patient while performing an orthopedic procedure on the patient.

### CROSS REFERENCE TO RELATED APPLICATIONS

This application claims priority to U.S. Patent Ser. No. 60/780,227, filed Mar. 8, 2006, which is hereby incorporated by reference herein.

### FIELD OF THE INVENTION

The field of the invention relates to devices for capturing emboli in a patient's blood vessels.

### BACKGROUND

A variety of devices have been used to capture emboli in blood vessels. These devices are evolving rapidly to improve safety, reliability, convenience, and cost. In general, such devices are highly specific to an intended application because the vascular system is complex, with many different sizes, shapes, and flow conditions. Also, these devices are often introduced through small cuts in the patient and must be navigated through the vasculature consistently and quickly, such that the shape and design of the device limits its use to a specific clinical indication for the particular method of introduction.

### SUMMARY OF THE INVENTION

Introduction

Vascular debris is formed in and/or is released into the venous system not only after, but even during, lower extremity orthopedic surgery, particularly (though not exclusively) knee and hip surgery. This debris often travels through the venous system through the right heart to the lungs. This debris, referred to as emboli herein, typically includes thrombotic material and fat, forms during blood stasis and/or is released from the marrow as a result of its manipulation during surgery. These emboli contribute to peri-operative hypoxia and other pulmonary and systemic complications that can result from these procedures.

Moreover, the post-operative cognitive dysfunction (POCD) that occurs in a large minority of patients undergoing these orthopedic and other surgical procedures is believed in part to be related to these emboli. Some of the material injures the lungs, and the substances released from the injured lungs are believed to injure the brain and contribute to or cause the cognitive dysfunction. But some of the smallest embolic particles are believed to cross through the pulmonary vasculature and travel to the brain, directly injuring it and contributing to cognitive dysfunction. How this material crosses from the venous system to the left side of the heart and then travels to the brain (and other vital organs, such as the kidney) is not clear. It does not only pass through atrial septal defects or patent foramen ovale; POCD occurs in many patients without such intra-cardiac defects. Intrapulmonary shunting may allow the passage of the emboli from the venous to arterial side. Evidence that the debris does, by whatever mechanism, travel from the venous system to the arterial circulation includes transcranial Doppler evidence of large amounts of debris traveling in intracranial arteries during hip and knee surgery (particularly after release of the femoral vein tourniquet often used during these operations), transesophogeal evidence of debris passing through not only the right but left side of the heart during these procedures, MRI evidence of cerebral infarcts following lower extremity orthopedic surgery, and autopsy evidence of lipid emboli in the brain among patients who die shortly after knee or hip replacement. Regardless of exactly how the debris gets from the venous system to the arterial system, there is increasing evidence that it does, and that it contributes to the POCD seen in many patients.

### Overview of Select Embodiments

What is needed is a device that will prevent lung and brain damage resulting from emboli. Disclosed herein is a tool suitable for placement within a vein during orthopedic or other surgical procedures to trap debris such as emboli for either removal from the body or its dissolution, as well as other related methods of preventing pulmonary and other complications resulting from embolization of said debris.

This device would be introduced into the venous system most commonly via the femoral vein and be deployed in the femoral, iliac, or other vein and capture emboli traveling from more distally in the venous system to the right heart and lungs, reducing or preventing pulmonary embolization and any POCD that these emboli may contribute to. The device can also be introduced into upper extremity veins during surgery on an arm.

In some embodiments, the device has a trap that includes a filter and optional filter support that would either be connected to a sheath, or to an actuating element connected to the filter inserted through the vascular sheath. The support has a first configuration in which the filter is collapsed (un-deployed, or not actuated) and a second configuration in which the filter is expanded (deployed, or actuated). The actuation device controls transition of the filter between the first configuration and the second configuration.

In another embodiment, the device has an occlusion balloon used to cause stasis in a vein, preventing blood flow and the movement of the emboli back to the heart and lungs. The occlusion balloon would function as an embolic trap, and allow the later suctioning of the static column of blood containing the various kinds of emboli generated by the surgical (or percutaneous) procedure.

In another embodiment, the invention pertains to a method for removing debris and/or dissolution of aggregated thrombus particulate closer to or at the site of formation, and not just downstream of the procedure.

### BRIEF DESCRIPTION OF THE FIGURES

FIG. 1 depicts an embodiment of a sheath for use with an embolic trap;

FIG. 2A depicts an obturator with an occlusion balloon;

FIG. 2B depicts the embodiment of FIG. 2A with the balloon deflated;

FIG. 2C depicts an alternative embodiment of the balloon of FIG. 2A;

FIG. 3A depicts an occlusion balloon being passed through a catheter for deployment in a vein;

FIG. 3B depicts the balloon of FIG. 3A inflated and deployed to trap blood flowing in the vein while suction removes the trapped blood;

FIG. 4A depicts a side view of an obturator comprising an occlusion balloon;

FIG. 4B depicts a cross-sectional view of the embodiment of FIG. 4A with arrows indicated the flow of blood into the obturator;

FIG. 5 depicts a blood filter deployed in a blood vessel;

FIG. 6A depicts a blood filter in a collapsed state being placed into a blood vessel;

FIG. 6B depicts the filter of FIG. 6A in an expanded state deployed in the blood vessel;

FIG. 6C depicts an alternative embodiment of a blood filter;

FIG. 7A depicts a blood filter on an obturator being passed through a catheter;

FIG. 7B depicts the filter of FIG. 7A in an expanded position;

FIG. 7C is an end view taken along the arrow 7C in FIG. 7B; and

FIG. 8 depicts an embodiment of a bundle of fibers on a support.

### DETAILED DESCRIPTION OF PREFERRED EMBODIMENTS

An embolic trap device may be used to trap emboli and stop its migration and consequent damage caused by blockage of a blood vessel by the embolic debris. Emboli are often generated during orthopedic procedures and can cause post-operative cognitive dysfunction, although this fact is not conventionally appreciated. In some methods, an embolic trap device is deployed in combination with orthopedic procedures to prevent the lung and potential brain damage that these procedures may cause. Although various procedures are known for treating emboli in other situations, those procedures are not suited for use in orthopedic procedures because the anatomy is different. Further, orthopedic surgeons are not familiar with embolic taps and removal devices because such devices are not used in peripheral orthopedic surgeries.

One challenge is that conventional embolic traps do not work with the small debris generated by orthopedic procedures. Orthopedic procedures, in general, produce emboli that are distinct from, and may be much smaller than, those generated by other procedures. Bone and bone marrow have a structure and composition quite distinct from other tissues. Much bone, for instance, is cancellous with many small pores. Some of these pores contain fat, as does bone marrow in the middle regions or marrow of many bones. This fat can be dislodged by surgical procedures, and flow into the blood in the form of small aggregates, also referred to as fat blebs herein. These fats blebs can and do embolize and can cause lung and brain damage. Removing these blebs is not possible using filters configured for other procedures because they pass through the filters; the pore sizes of filters used for percutaneous revascularization procedures in the heart, carotid arteries, kidneys, etc., are much too large to capture the small particles of fat generated from orthopedic procedures. Moreover, most available filters are used in the arterial system and are designed for arteries much smaller than the veins in which the debris generated by orthopedic procedures such as hip and knee replacement arise from and travel through. Lastly, those existing devices are too complex to be used without flouroscopy; certain systems herein for removing embolic debris are so easy to use that flouroscopy is not needed. For instance, the system can be used in conjunction with a vascular sheath placed in the venous system without flouroscopy.

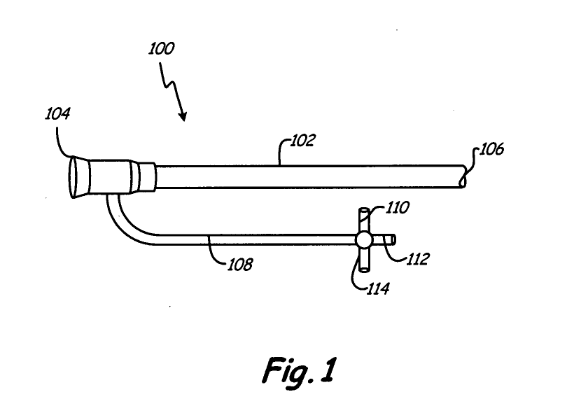

A first embolic trap is shown in FIGS. 1-3. FIG. 1 depicts a tube, or sheath 100 having catheter body 102, proximal end 104, distal opening 106, and side arm 108 having egresses 110, 112, and switch 114. Catheter body 102 has a lumen in fluid communication with distal opening 106 and the lumen of side arm 108, which lumen is in fluid communication with egresses 110 or 112, as controlled by position of switch 114. Sheath 100 has a one-way valve (not shown) near proximal end 104 so that suction applied through egress 110, 112 pulls fluid through distal opening 106. Proximal end 104 also has a diaphragm (not shown) that allows objects to be passed therethrough while maintaining a seal about the object.

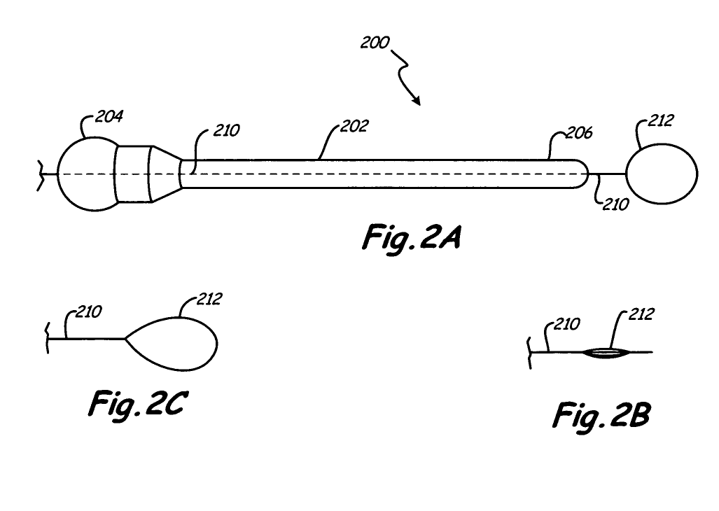

FIG. 2A depicts occlusive balloon assembly 200 with obturator 202 having proximal end 204, distal end 206, balloon 208, and hollow fill tube 210. FIG. 2B shows balloon 208 in an uninflated state. FIG. 2C shows alternate inflated balloon 212 having an elliptical inflated shape.

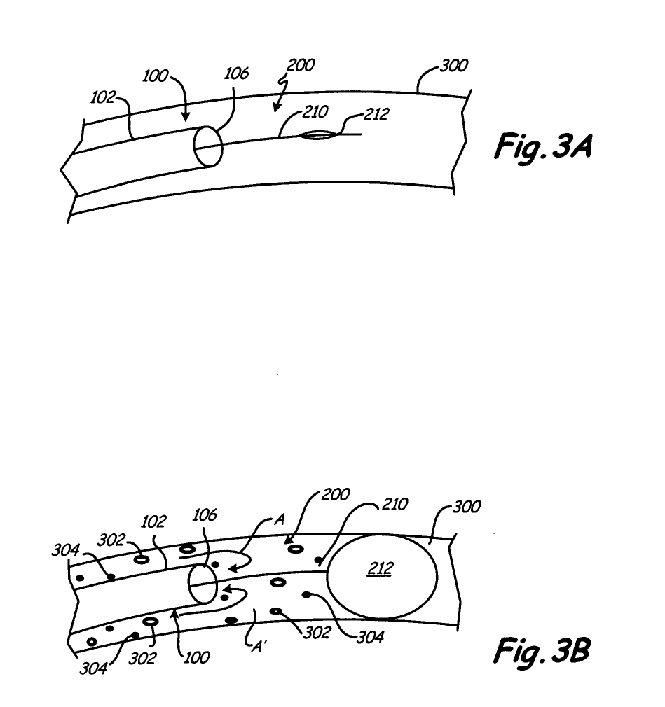

FIG. 3A shows balloon assembly 200 being advanced through sheath 100 inside vein 300. In FIG. 3B, balloon 212 is inflated and suction is being applied to pull fluid into distal opening 106, which creates a flow of fluid as indicated by the arrows A, A′. Emboli including thrombi 302 and relatively smaller fat blebs 304 are swept into distal opening 106.

In use, a conventional introducer is placed into the patient, with the introducer having a guidewire in its lumen. Sheath 100 is advanced over the introducer into the patient's blood vessel, and the introducer and guidewire are removed through the sheath's interior. Balloon assembly 208 is advanced through sheath 100 to a desired position by pushing obturator 202 until a desired length is advanced to indicate that the assembly is in position. Hollow fill tube 210 may be advanced as needed to place balloon 212 as needed. Once in place, balloon 212 is inflated using known means (not shown) such as fluid or gas. Balloon 212 is sized to expand against blood vessel 300's inner walls to form a substantially fluid-tight seal across the blood vessel. After the balloon has been deployed to seal the blood vessel, blood may be drawn through distal opening 106 to pull all emboli out of the vessel and into sheath 100. One technique for drawing blood is to fix a syringe to one of egresses 110, 112, position switch 114 to fluidly connect the chosen egress to the lumen of side arm 108, and pull the plunger of the syringe to create a vacuum. The vacuum causes the blood to flow as indicated in FIG. 3B. After drawing the blood, balloon 212 may be collapsed and withdrawn. Sheath 100 may be withdrawn and the introducer also withdrawn.

Alternatively, a vacuum pump or syringe pump may be affixed to one of the egresses to withdraw the blood. In some embodiments, the blood is withdrawn drawn on demand by a user. In other embodiments, the blood is also, or only, withdrawn according to a schedule, e.g., at a steady rate as determined by setting the syringe pump rate or vacuum pressure. A steady rate may be used to prevent complete stasis of the blood so as to reduce ischemia and avoid settling-out of some emboli. Another technique is to move sheath 100 or other hollow tube relative to the trap as blood is pulled into the sheath, so as to sweep the blood vessel. The sheath may be moved back and forth while withdrawing blood to thoroughly vacuum the blood vessel.

FIGS. 2 and 3 depict a hollow fill tube 210 for inflating balloon 212. The fill tube may be moved relative to obturator 202. Alternatively, the hollow fill tube may be fixed relative to the obturator. Alternatively, obturator may connect directly to a balloon or other trap, so that the fill tube is covered, or dispensed with altogether.

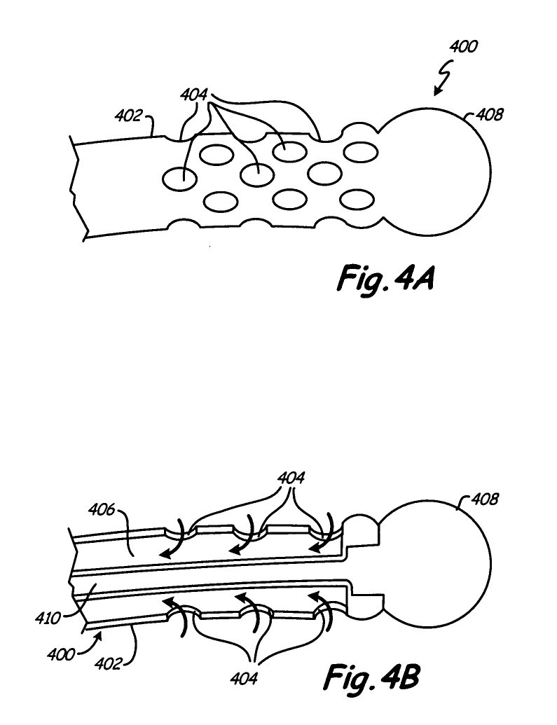

FIGS. 4A and 4B depict an alternative occlusion balloon assembly 400. Outer catheter 402 has a plurality of openings 404 in fluid communication with annulus 406. Balloon 408 is attached to the assembly and is inflatable or deflatable through inner lumen 410. Assembly 400 may be attached either directly to a sheath, as in sheath 100, or to another tool that passes through the sheath or other hollow tube, e.g., 402 may be an obturator as in obturator 202. In use, the assembly is deployed by inflating balloon 408 to occlude a blood vessel and suction is applied to inner lumen 410 to draw blood through openings 404 from whence it passes out of the body.

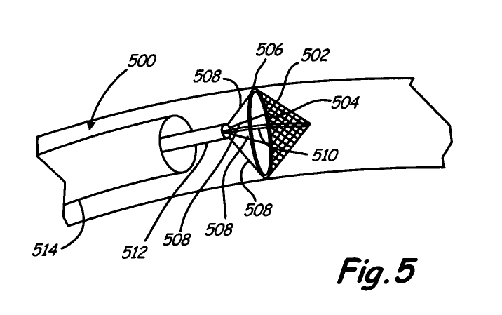

FIG. 5 depicts an alternative embolic trap assembly 500 that has a blood filter 502 with mesh 504, rim 506, struts 508, wire 510, and overtube 512. Struts 508 attach overtube 512 to rim 506, which is connected to mesh 504. Movement of wire 510 relative to overtube 512 causes struts to push or pull rim 506 to contract or expand. In use, assembly 500 is passed through a catheter as in catheter 514, past the end of the catheter, and the trap is deployed (expanded) by relative movement of wire 510 and overtube 512. Catheter 514 may be withdrawn to leave the assembly in place. Alternatively, catheter 514 may be left in place or an alternative tube may be passed over overtube 512 to bring openings of the tube into proximity to filter 502. A vacuum force is applied to pull blood into the catheter to pull emboli out of filter 502.

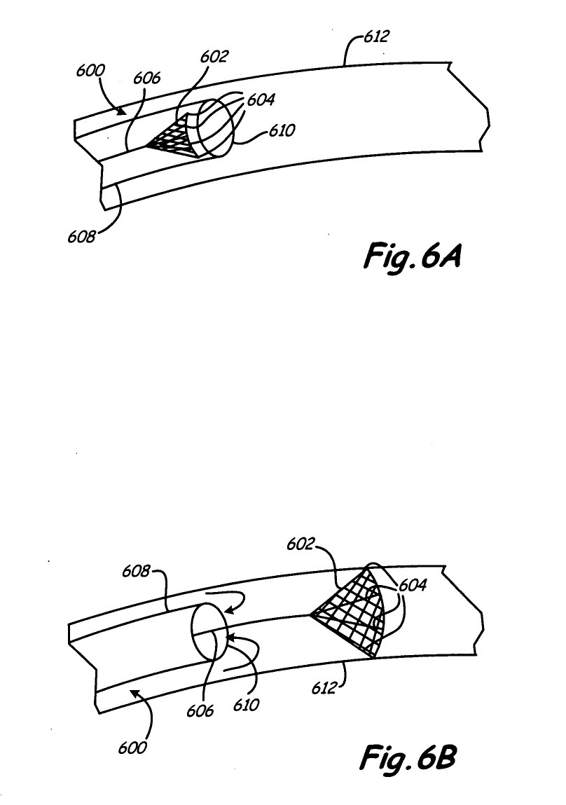

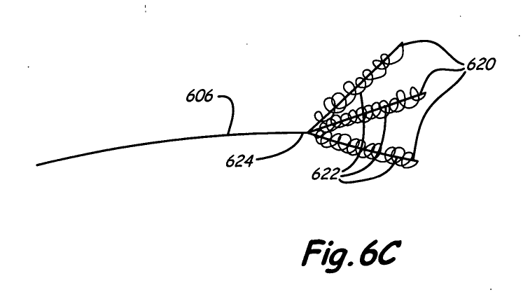

FIGS. 6A and 6B depict alternative expandable embolic trap assembly 600. Filter 602 has struts 604 that are biased to open relative to wire 606. A mesh is attached to struts 606 to filter blood. In use, catheter 608 is positioned in a blood vessel. Filter 602 is advanced through catheter 608 and beyond catheter opening 610 into the blood vessel 612. The filter is biased to open when not restrained by catheter 608, and opens to bridge blood vessel 612. Blood may be pulled through opening 610 into the interior of catheter 608 from whence it may pass out of the body. Filter 602 may be withdrawn through opening 610, which collapses the filter. Alternatively, the catheter may be withdrawn and the filter withdrawn from the body by pulling the wire and pulling the filter along the lumen of the blood vessel. An alternative embodiment depicted in FIG. 6C allows struts 620 to move freely relative to each other, in this case, by being attached only at base area 624. The struts 620 have fibrous material 620 attached thereto that serves to trap emboli, e.g., such as the lipophilic materials described below. This arrangement accommodates a wide variety of vessel shapes and diameters. Only a few struts are shown in FIG. 6C for clarity: many more struts could be used to better assure thorough filtering of the blood. Alternatively, the struts may be loosely interconnected such that they can move freely relative to each other without being completely independent in such movement.

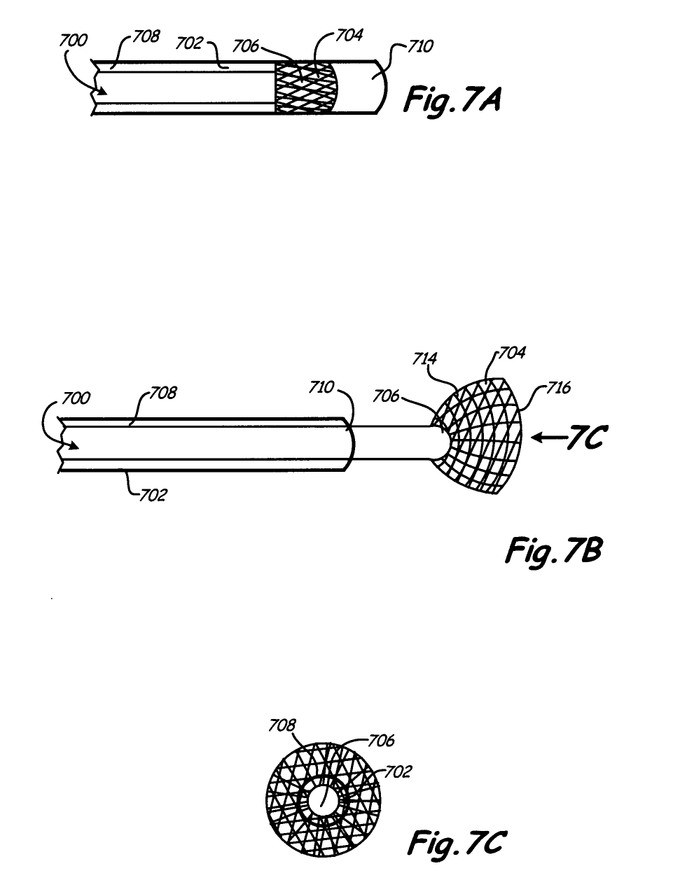

FIGS. 7A-7C depict a self-expanding embolic trap affixed to a catheter passed through an outer catheter. Specifically, assembly 700 has evacuation catheter 702 with self-expanding filter 704 affixed thereto, with evacuation catheter opening 706 being unobstructed. In use, assembly 700 is advanced through outer catheter 708, with filter 704 being restrained in a state of collapse (undeployed) by walls of outer catheter 708. As assembly 700 is passed through outer catheter opening 710, it expands. In use, an introducer or a sheath such as sheath 100, or a catheter is advanced to a desired position in a blood vessel. The assembly 700 is advanced therethrough and out of opening 710, with filter 704 expanding thereupon to bridge a blood vessel. Blood flows past the filter and is trapped on proximal side 714 located between the filter and evacuation catheter 702 or on the distal side 716 located at the end of filter 704 farthest from the evacuation catheter 702. If the trapping is performed on the proximal side, either evacuation catheter 702 and/or outer catheter 708 may have openings (not shown) for drawing-in blood and emboli. If trapping is performed on distal side 716, blood and emboli may be drawn in through opening 706. A vacuum is used to draw the blood in, while keeping the proximal end of evacuation catheter 702 closed essentially prevents blood from passing into the catheter. The orientation of the assembly 700 relative to blood flow in the vessel normally determines which side emboli are trapped on, since the flowing blood tends to deposit emboli in the filter. Alternatively, both sides of the filter may be used to trap emboli by pushing/pulling the filter through the vessel. The assembly 700 may be removed from the patient by withdrawing it into outer catheter 708, with filter 702 collapsing (undeploying) as it is pulled therein. Alternatively, outer catheter 708 may be removed after filter 702 is placed and filter 702 subsequently withdrawn without passing through the same.

The embodiment of FIGS. 7A-7C is useful for venous applications as described herein and is also particularly suited to certain arterial applications, e.g., in a femoral or iliac artery. In conventional practice, an iliac occlusion is often treated with a percutaneous revascularization procedure. Such occlusions, including total or subtotal, are at a high risk of embolizing and for that reason are usually approached via the contralateral femoral artery so that when embolization does occur, the equipment can be advanced further down the leg and used to treat the embolus. As provided herein, however, such procedures may be accomplished without accessing the contralateral femoral. A sheath may be associated with an embolic trap, e.g., a filter or a funnel-shaped filter, such that medical tools or equipment may be passed through the sheath and through the filter. For this application, the filter may have a central opening through which central lumen of the sheath projects that allows passage of catheters and other tools through the filter, allowing for the capture and removal of emboli, either as described in these Figures (e.g., FIGS. 7A-7C) or using other motifs disclosed herein. Examples of diameters for the opening sized to allow passage of medical tools are from about 0.05 to about 0.5 inches; artisans will immediately appreciate that all the ranges and values within the explicitly stated ranges are contemplated, e.g., about 0.1 to about 0.2 inches.

Such a sheath may be introduced into the artery downstream of the occlusion, e.g., through percutaneous access of the femoral artery on the same side (ipsilateral side) as the occluded iliac artery. Then emboli created by treating the occlusion will flow from the occlusion down towards the filter, where they will be trapped and/or enter the sheath. Such debris may be removed periodically during or after the procedure, for instance, the filter may be undeployed or otherwise withdrawn into the sheath after the procedure along with emboli therein. The emboli may thus be removed from the body. Alternatively, other embolic traps as described herein may be used for such applications.

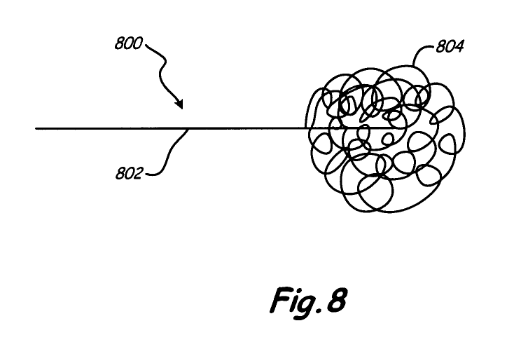

FIG. 8 depicts embolic trap 800 comprising fibers 804 mounted on support 802. As depicted, the fibers form a bundle at the end of the support. Such a bundle may be delivered through a catheter in a collapsed state and can then be opened when they are not being held taut, they will then expand to fill a space such as a blood vessel without unduly straining the same. The support may be a wire, guidewire, obturators or other suitable structure. The fibers may be attached by conventional means, e.g., adhesive. The fibers may be, e.g., comprised of lipophillic fibers or materials that preferentially attract and bind lipid, or fat. For instance, hydrophobic (i.e., lipophillic) fibers include homopolymers or copolymers of, e.g., polyolefin fibers, polypropylene, polyethylene, poly(ethylene terephthalate), polytetrafluoroethylenes, halogenated polymers or olefinic polymers, and fibers treated to be hydrophobic, e.g., with waxes, fatty acids, and/or as in U.S. Pat. Nos. 3,926,548, 5,958,806, 6,177,191, 6,287,689, 6,524,349, and 6,811,716, each of which are hereby incorporated by reference to the extent they do not contradict what is explicitly disclosed herein. And various biocompatible materials may be used to make the fibers, e.g., polyesters, nylons, dacrons, polylactides, or polyglycolics.

Some embodiments relate to the use of an obturator. This terms refers to a flexible member that resists kinking when flexed to pass within a blood vessel, yet having enough stiffness to be easily manipulated by hand. Obturators may be manually pushed—without kinking—by a user to ease them into the blood vessel. These may be made of, e.g., flexible plastic, e.g., polyethylene, polypropylene, polyurethane, or polytetrafluoroethylene. An obturator typically has a maximum outer diameter smaller than the internal diameter of the sheath in which is placed. The ends of obturators are typically rounded to ease passage through a blood vessel. The obturator is designed to be flexible and prevent compression or collapse or kinking of the sheath if, for example a thin-walled sheath is placed in the femoral vein and the hip is flexed, as it has to be during some types of orthopedic surgery.

Obturators may be combined with an embolic trap. Thus some embodiments of obturators are flexible plastic rods with a length of about 6-40 inches; artisans will immediately appreciate that all the ranges and values within the explicitly stated ranges are contemplated, e.g., 6-24 inches, 10 inches, or 30 inches. In some embodiments, the obturator may be reversibly bent as much as 90 degrees without breaking or permanently deforming. Examples of outer diameters are about 0.05 to about 0.5 inches; artisans will immediately appreciate that all the ranges and values within the explicitly stated ranges are contemplated, e.g., about 0.1 to about 0.2 inches. Such obturators may be solid or have a lumen therein for passage of a wire or guidewire. The embolic trap may be affixed to the obturator or be connected indirectly to the obturator. Further, obturators may be used to deploy an embolic trap without a guidewire, with the term guidewire referring to thin wires used to guide a medical device through the vasculature to place the medical device, typically with a diameter of less than about 0.080 inches. The wires used in conjunction with a conventional introducer are thus not guidewires, and hollow wires or tubes used to fill an occlusion balloon do not have to be guidewires.

While certain embodiments of traps are described as self-expanding or self-deploying, alternative embodiments are traps that deploy by manipulating a wire to deploy or undeploy the trap, e.g., a balloon or a filter. Certain wire-based techniques are described herein, and techniques as described elsewhere may be used as needed, e.g., as in U.S. Pat. Nos. 5,053,008, 5,766,191, 6,168,579, 6,371,970, and 6,652,557, which are hereby incorporated by reference herein to the extent they do not contradict what is explicitly disclosed herein.

The term embolic trap includes not only filters and the like that directly trap emboli, but also reversibly occlusive devices used in combination with a means for evacuating any emboli that accumulate in a blood vessel. Certain embodiments relate to occlusive balloons. Occlusive balloons may be fillable using a tube, e.g., a metal hollow wire or a plastic fill-tube. An occlusive balloon blocks essentially all blood flow when inflated or deployed, and occludes a blood vessel. A balloon that is inflatable and deflatable is reversibly occlusive. A means for suction may include a tube configured to have suction (or other vacuum) applied in its lumen to cause blood or emboli to enter the suction device, allowing the removal of embolic debris trapped by the occlusive balloon. Certain embodiments thus relate to occlusive balloons. Occlusive balloons may be fillable using, e.g., a tube, e.g., a metal hollow wire or a plastic hollow fill-tube.

Alternatives to balloons may be used, with such occlusive devices effectively blocking blood flow when deployed in a blood vessel. For instance, instead of a filter as depicted herein, a solid plug or membrane that does not allow fluid passage may be deployed.

Certain embodiments relate to embolic traps that filter the blood. Exemplary traps have been described. Alternatives include meshes, sponges, fibrous mats, interconnected fibrils, and porous sacs. The filters may be configured to trap essentially all emboli that are generated in orthopedic procedures, or subsets thereof. Emboli from such processes include thrombi, aggregates of red blood cells or platelets joined by fibrin, fibrin clots, plaques as are known to accumulate on blood vessel walls as well as fragments thereof, bone fragments, cartilage fragments, and also fat blebs. The fat blebs tend to be smaller than the other emboli, with sized that are only somewhat larger than red blood cells. Accordingly, filters may have be configured to pass emboli (or not) according to size. A filter may be comprised of lipophillic fibers or materials that preferentially attract and bind lipid, or fat. Emboli of all sizes can cause damage, and different types of procedure can produce varying ranges and types of emboli.

Thus different size cut-offs for the pore size of filters may be used as needed. A range of cut-offs for filters may this vary from about 5 microns to about 2000 microns; artisans will immediately appreciate that all ranges and values between the explicitly stated values are contemplated, e.g., filters that trap particles larger than about 1000 microns, about 500 microns, about 200 microns, about 100 microns, about 50 microns, about 20 microns, about 15 microns, about 10 microns, or about 8 microns.

Examples of meshes for filters that may be sized to various cut-offs are porous weaves or fabrics, sheets with holes, e.g., made by lasers to a particular size, coiled strands, or braids. Materials include, e.g., polyesters, poly(ethylene terephthalate) (PET), expanded polytetrafluoroethylene, nylons, dacrons, polylactides, or polyglycolics. Alternatively, fine wires may be made into a mesh with a defined pore size. Again, fibers can be altered to be lipophillic to preferentially bind lipid or fat particles and capture such debris even if the effective pore size is bigger than the size of the debris.

In some applications, an embolic trap is deployed relatively close to a point of entry into a patient. For instance, if the trap is to be placed in the iliac vein with the femoral vein as an access site, the trap must be advanced a distance of approximately six inches into the patient. Or, for instance, if the same access site is used but the inferior vena cava is the desired site for trap deployment, a distance of as much as about 24 inches may be required. Deployment of the embolic trap in the inferior vena cava might be particularly beneficial for capturing embolic debris generated during orthopedic procedures such as those involving the spine. In either case, embodiments as in FIGS. 1 to 7 may be adapted, e.g., by using a catheter and/or wires of appropriate length, strength, and flexibility. For relatively longer distances, a flexible catheter may be steered from the introducer up into the desired site, e.g., the inferior vena cava. A guidewire (e.g., 0.014 inch diameter) is passed through the catheter. The embolic trap is guided along the guidewire and passed beyond the end of the catheter and deployed to occlude the blood vessel. Suction is applied through the catheter to remove blood and emboli.

Alternatively, after advancing the catheter, an embolic trap on a wire (e.g., a fill tube in the case of a trap using an occlusive balloon) is advanced therethrough, is passed beyond the tip of the catheter, and deployed in the blood vessel without the use of a separate guidewire. The choice of which technique to use depends in part on factors dictated by the particular site of deployment and of access, the complexity of navigating to the site, the tortuosity of the vasculature between the site of entry and desired deployment site. Some examples of guidewire-based devices are provided in, e.g., U.S. Pat. Nos. 5,540,707; 5,935,139; 6,050,972; 6,371,970; 6,875,193; 6,800,080; which are hereby incorporated by reference herein to the extent they do not contradict what is explicitly disclosed herein.

The embolic trap may be deployed as needed to trap emboli. In general, deployment in a vein advantageously captures the emboli as they enter the body's vasculature and circulate to the right heart and lungs. The embolic trap is deployed at a position between the heart and the venous beds that receive blood from the site of the operation or procedure. The blood flowing from the operative area passes into or through the trap and the emboli are removed.

The iliac vein is one site for deployment of an embolic trap. The venous blood in the leg returns from the common femoral vein, which feeds the iliac vein, which in turn feeds the inferior vena cava. In the case of an operation in the leg, therefore, the venous blood passes through leg veins, and the trap may be deployed in any vein that is calculated to receive some or most of the emboli generated by the operation. In many cases, this will be the femoral vein or the iliac vein. Essentially all of the blood from the leg flows through the iliac vein, so that deployment in the iliac vein is generally effective for leg operations, including both knee or hip procedures. Hip or knee replacement are types of hip or knee procedures, as well as certain other reparative hip and knee procedures. The inferior vena cava is also appropriate as a site for deployment, and similarly provides a single position to filter all the venous blood not only from the peripheral circulation but also blood with debris generated during spine surgery. Accordingly, some embodiments provide for an embolic trap to be placed in the inferior vena cava. Embolic trap placement in the inferior vena cava is often a strong option for orthopedic surgeries directed to a spinal area. Spinal area surgeries that are below the heart are suited to have embolic debris removed by placement of the device in the inferior vena cava.

Therapeutic agents, e.g., drugs, may be used in combination with the methods and devices described herein. In some embodiments, tissue plasminogen activator, urokinase, heparin, bivalirudin, tenecteplase, or other thrombolytic or fibrinolytic drugs may be used. The drugs may be attached to elements of the filter itself or be administered systemically, locally, or through a catheter for delivery to a blood vessel.

As explained, therefore, some embodiments are directed to a method of treating a patient comprising deploying an embolic trap to remove emboli from a vein or other blood vessel of the patient while performing an orthopedic procedure on the patient. Such orthopedic procedure may be, e.g., a knee surgery or hip surgery, or spine surgery. The trap may be placed, e.g., in a femoral vein, iliac vein or inferior vena cava of the patient. The trap may be placed between the site of the orthopedic procedure and the patient's right heart chamber. Some embodiments are those comprising introducing the trap into the patient in a collapsed position and expanding the trap to deploy it in the venous blood vessel. The trap may comprise an occlusion balloon placed in the vein to occlude the vein and a tube for removing blood in the vein. Some embodiments are directed to removing blood and/or emboli proximal of the trap and/or in the trap by suction through the tube. The balloon may be integral to the tube, e.g., as in a support tube.

In some embodiments, an occlusion balloon is fillable through a fill tube that passes through an obturator, with the method involving placing a catheter, passing at least a portion of the obturator and balloon through a catheter into the blood vessel, placing the balloon at a desired position in the vein, inflating the balloon through the fill tube to block the flow of blood in the vein, and after the flow of the blood in the vessel is blocked: starting the orthopedic procedure, and removing blood and the emboli from the vein through the catheter. Alternatively, the orthopedic procedure may be started before the filter or balloon is deployed according to any of the embodiments herein. A catheter refers to a medical tube. Such tubes may be equipped as needed with valves, one-way valves, seals for passing instruments, guidewires, and other assemblies.

In some embodiments, an occlusion balloon is fillable through a fill tube, and its use may involve placing a catheter, passing the balloon through the catheter into the blood vessel, placing the balloon at a desired position in the vein, inflating the balloon through the fill tube to block the flow of blood in the vein, and (optionally after the flow of the blood in the vein is blocked) starting the orthopedic procedure, and removing blood and the emboli from the vein through the catheter.

In some embodiments, an occlusion balloon is fillable through an obturator lumen, and its use may involve placing a catheter in a blood vessel, passing at least part of the obturator and balloon through the catheter into the blood vessel, placing the balloon at a desired position in the vein or other blood vessel, inflating the balloon through the lumen of the obturator to block the flow of blood in the vein or other blood vessel, and (optionally after the flow of the blood in the vein is blocked), starting the orthopedic procedure, and removing blood and the emboli from the vessel through the catheter.

In some embodiments, an occlusion balloon (or a filter) is integral with a support tube, and a method of placement comprises placing a catheter into a blood vessel, passing the support tube and/or balloon through a catheter and into the blood vessel, placing the balloon at a desired position in the vein, inflating the balloon through a fill tube to block the flow of blood in the vein, and (optionally after the flow of the blood in the vein is blocked) starting the orthopedic procedure, and removing blood and the emboli from the vein through the support tube and/or the catheter, or other tube placed for that purpose.

And the trap, in some embodiments, comprises a filter that filters the emboli and allows blood fluid to pass. The filter can comprise a mesh of fibers that allow blood to pass.. And the filter may comprises a bundle of fibers.

In some embodiments, the filter is deployable by moving a wire that passes through an obturator, and its use may involve placing a catheter into a blood vessel of the patient, passing at least part of the obturator and filter through the catheter into the blood vessel, placing the filter at a desired position in the vein, moving the wire to deploy the filter to filter blood flowing in the vein or other blood vessel, and, at a suitable point, starting the orthopedic procedure, and removing the emboli from the vein through the catheter and/or by withdrawal of the filter.

In some embodiments, the filter is deployable by moving a wire, and its use may involve placing a catheter into a blood vessel of the patient, passing the filter through the catheter into the blood vessel, placing the filter at a desired position in the vein or other blood vessel, and moving the wire to deploy the filter to filter blood flowing in the blood vessel, and, at a suitable point, starting the orthopedic procedure, and removing blood and the emboli from the vein through the catheter and/or by withdrawal of the filter.

In some embodiments, the filter is integral with a support tube, and placement may involves placing a catheter into a blood vessel of the patient, passing the filter and optionally at least part of the support tube through the catheter and into the blood vessel, placing the filter at a desired position in the vein, and deploying the filter to filter blood flowing in the vein, and starting the orthopedic procedure, and removing blood and the emboli from the vein through the support tube and/or the catheter and/or by withdrawal of the filter.

In some embodiments, an embolic trap comprises a flexible plastic obturator having a length of between about 6 inches and about 30 inches and an occlusion balloon or blood filter. The blood filter may be attached directly to the obturator. The blood filter may comprise a bundle of hydrophobic fibers. The balloon may be attached to a distal end of the obturator and fillable through a fill tube in the obturator. The obturator may comprise a lumen and the occlusion balloon or the blood filter may be mounted on a wire that is movable within the lumen.

The device, while described in general herein with reference to the venous system, may also be adapted for placement within arteries prevent the embolization of atherosclerotic, lipid, thrombotic, and other debris from blocking the flow of blood and other bodily fluids to downstream organs and other arteries.

The invention has been described with respect to particular embodiments having various features. The features of these embodiments may be mixed-and-matched to form other combinations as guided by the need to make an operable device.

What is claimed:

1. A method of treating a patient comprising deploying an embolic trap to remove emboli from a vein of the patient while performing an orthopedic procedure on the patient.

2. The method of claim 1 wherein the orthopedic procedure is knee surgery, hip surgery, or spine surgery.

3. The method of claim 1 comprising placing the trap in a femoral vein, iliac vein, or inferior vena cava of the patient.

4. The method of claim 1 comprising placing the trap between the site of the orthopedic procedure and the patient's right heart chamber.

5. The method of claim 1 comprising introducing the trap into the patient in a collapsed position and expanding the trap to deploy it in the venous blood vessel.

6. The method of claim 1 wherein the trap comprises an occlusion balloon placed in the vein to occlude the vein and a tube for removing blood in the vein.

7. The method of claim 6 further comprising removing blood and/or emboli proximal of the trap and/or in the trap by suction through the tube.

8. The method of claim 6 wherein the balloon is integral to the tube.

9. The method of claim 6 wherein the balloon is fillable through a fill tube that passes through an obturator, and further comprising:
placing a catheter into a blood vessel of the patient,
passing the obturator and balloon through the catheter into the blood vessel,
placing the balloon at a desired position in the vein,
inflating the balloon through the fill tube to block the flow of blood in the vein,
starting the orthopedic procedure, and
removing blood and the emboli from the vein through the catheter.

10. The method of claim 6 wherein the balloon is fillable through a fill tube, and further comprising:
placing a catheter into a blood vessel of the patient
passing the balloon through the catheter into the blood vessel,
placing the balloon at a desired position in the vein,
inflating the balloon through the fill tube to block the flow of blood in the vein,
starting the orthopedic procedure, and
removing blood and the emboli from the vein through the catheter.

11. The method of claim 6 wherein the balloon is fillable through an obturator lumen, and further comprising:
placing a catheter into a blood vessel of the patient,
passing the obturator and balloon through the catheter into the blood vessel,
placing the balloon at a desired position in the vein,
inflating the balloon through the lumen of the obturator to block the flow of blood in the vein,
starting the orthopedic procedure, and
removing blood and the emboli from the vein through the catheter.

12. The method of claim 6 wherein the balloon is integral with a support tube and fillable with a fill tube, and further comprising
placing a catheter into a blood vessel of the patient,
passing the balloon through the catheter and into the blood vessel,
placing the balloon at a desired position in the vein,
inflating the balloon through the fill tube to block the flow of blood in the vein,
starting the orthopedic procedure, and
removing blood and the emboli from the vein through the support tube.

13. The method of claim 1 wherein the trap comprises a filter that filters the emboli and allows blood fluid to pass.

14. The method of claim 13 wherein the filter comprises a mesh of fibers that allow blood to pass.

15. The method of claim 13 wherein the filter comprises a bundle of hydrophobic fibers.

16. The method of claim 13 wherein the filter is deployable by moving a wire that passes through an obturator, and further comprising:
placing a catheter into a blood vessel of the patient,
passing the obturator and filter through the catheter into the blood vessel,
placing the filter at a desired position in the vein,
moving the wire to deploy the filter to filter blood flowing in the vein,
starting the orthopedic procedure, and
removing the emboli from the vein through the catheter.

17. The method of claim 13 wherein the filter is deployable by moving a wire, and further comprising:
placing a catheter into a blood vessel of the patient,
passing the filter through the catheter into the blood vessel,
placing the filter at a desired position in the vein,
moving the wire to deploy the filter to filter blood flowing in the vein,
starting the orthopedic procedure, and
removing blood and the emboli from the vein through the catheter.

18. The method of claim 13 wherein the filter is integral with a support tube, and further comprising:
placing a catheter into a blood vessel of the patient,
passing the filter through the catheter and into the blood vessel,
placing the filter at a desired position in the vein,
moving the wire to deploy the filter to filter blood flowing in the vein,
starting the orthopedic procedure, and
premoving blood and the emboli from the vein through the support tube.

19. An embolic trap comprising a flexible plastic obturator having a length of between about 6 inches and about 30 inches and an occlusion balloon or blood filter.

20. The trap of claim 19 comprising the blood filter, wherein the blood filter is attached directly to the obturator.

21. The trap of claim 19 comprising the blood filter, wherein the blood filter comprises a bundle of hydrophobic fibers.

22. The trap of claim 20 comprising an occlusion balloon, wherein the balloon is attached to a distal end of the obturator and is fillable through a fill tube in the obturator.

23. The trap of claim 20 wherein the obturator comprises a lumen and the occlusion balloon or the blood filter is mounted on a wire that is movable within the lumen.

24. A method of treating a patient comprising using a percutaneous access through a femoral artery to deploy an embolic trap in the femoral artery or an iliac artery and to remove emboli with the trap while performing a procedure on the iliac or the femoral artery of the patient.

25. The method of claim 24 wherein the embolic trap comprises a filter with an opening to a lumen of a percutaneous catheter, with the lumen being sized to allow passage of medical tools.
